(19) United States
(12) Patent Application Publication
(10) Pub. No.: US 2017/0296471 A1
(43) Pub. Date: Oct. 19, 2017

(54) UNIQUE HIGH SPEED TABLET DRILLING LASER SYSTEM

(71) Applicant: SCANTECH LASER PVT. LTD., Navi Mumbai (IN)

(72) Inventor: Naren Shriram Shikarkhane, Navi Mumbai (IN)

(21) Appl. No.: 15/426,068

(22) Filed: Feb. 7, 2017

(30) Foreign Application Priority Data
Apr. 13, 2016 (IN) 201621013054

(51) Int. Cl. A61K 9/20; B23B 35/00; A61K 9/28; G02B 26/08

(52) U.S. Cl. CPC A61K 9/2072; G02B 26/0816; A61K 9/2095; A61K 9/28; B23B 35/00

(57) ABSTRACT

An apparatus for high speed laser drilling of tablets, particularly controlled-release tablets is disclosed. The apparatus comprises of a rotary disk (118) with radial slots (401) to hold the tablets in position with help of centrifugal force. The apparatus further comprises a laser system (120) configured to fire a laser beam in order to draw a line of required length on tablets to drill a precise hole at high speed. The laser system (120) is enabled to draw a line on tablet at a speed equal to that of rotational speed of tablets on the rotary disk (118).

### CROSS-REFERENCE TO RELATED APPLICATIONS AND PRIORITY

The present application does claims priority from Indian Patent Application No. 201621013054 dated 13 Apr. 2016, the entirety of which is incorporated herein by a reference.

### TECHNICAL FIELD

The present invention in general relates to a tablet drilling apparatus. More particularly, the present invention relates to a laser system for drilling hole into controlled-release tablets for mechanical time-release of medicine, with simplified operation and improved accuracy.

### BACKGROUND

The laser drilling for controlled-release tablets is well known in prior art. The controlled-release tablets are also known as sustained-release tablets due to their property of mechanical time-release of medicine. The controlled-release tablets (hereinafter referred as “tablets”) may be monolayer or bilayer tablets. The tablets have a surface coating that prevents immediate dissolution of medicine once ingested in a digestive track. It is to be noted that when a small hole is drilled in a tablet, the medicine is forced out of the hole at a constant rate. The surface coating of the tablet acts like a semipermeable membrane that absorbs fluid (e.g. gastrointestinal fluid) and pushes out the medicine through the hole. In case of bilayer tablets, the first medicine is dissolved first and the second medicine is dissolved later. It is important to release the medicines in given order and over a given period. To control the mechanical time-release accurately, the hole in the tablet is required to be drilled with high degree of precision.

In the existing art, few machines are available that facilitates drilling of holes in the tablet. These machines use conveyor belts or rotary disks for transporting the tablet to drilling means to drill a hole into the tablet mechanically or electronically. These machines also use a clamping means to hold the tablet in right position before the hole is drilled. Since the clamping means hold a single tablet in a slot of the clamping means at a time, rate of tablets processed per hour is very low. To increase this rate, these machines have high speed and to satisfy the precision requirements, these machines use laser firing technique.

The hole to be drilled in the tablets usually has a diameter below 1 mm. To drill the hole, the aforementioned machines available enables drawing a circle of small diameter with the help of galvo scanners with 2XY beam, deflecting mirrors, special software and hardware. Due to fast motion of the tablets, the circles drawn are not perfect and can become elliptical or oval. A software compensation is required to compress or elongate the circles in order to match and overcome the effect of motion in opposite direction. Therefore, an active feedback regarding speed of the tablet for working out the amount of compression of the drawn circle is required. The feedback may be obtained via an encoder adapted to sense the speed and act accordingly. A “On the Fly” software/hardware is used in some prior arts to achieve the precision in drilling. However, the “On the Fly” software/hardware makes the overall system costly and time consuming.

Many times more than one hole must be drilled in the tablet. In such scenarios, the existing machines may be employed to draw two or more circles, each circle representing a single hole in the tablet. This increases the overall processing time. Further, the existing machines need to drill the two holes such that the distance between two holes is accurate. Further, since the fast motion of the tablets may change the circularity of the hole, the existing machines need to verify whether the hole is drilled with expected circularity. This demands additional software and scanners or image processing equipment and sensors.

Most of the time the tablets are bilayer with two different colours, however, the hole is required to be drilled only on one side of the tablet. Therefore, in order to drill the hole on correct layer/side, the existing machines must first sort out the tablets, check the orientation of the tablets with the help of scanners or cameras and reject the tablets oriented wrongly. The rejected tablets are returned to the tablet holder. From the tablet holder, the tablets are again fed into the machine. The above process is repeated until the tablets are all oriented properly. A separate sensor is employed in order to signal a laser system whether or not a beam is required to be fired depending on the correct or incorrect orientation of the tablets. This in turn increases the processing time and reduce the output rate.

### SUMMARY

Before the present apparatus and its components are described, it is to be understood that this disclosure is not limited to the particular apparatus and its arrangement as described, as there can be multiple possible embodiments which are not expressly illustrated in the present disclosure. It is also to be understood that the terminology used in the description is for the purpose of describing the particular versions or embodiments only, and is not intended to limit the scope of the present application. This summary is not intended to identify essential features of the claimed subject matter nor is it intended for use in detecting or limiting the scope of the claimed subject matter.

In one aspect, the present invention pertains to an apparatus for drilling holes into a plurality of tablets. The apparatus comprises a rotary disk comprising a plurality of radial slots. The plurality of radial slots facilitates in sorting of a plurality of tablets received through a hopper on the rotary disk. Each radial slot is adapted to hold one or more tablets of the plurality of tablets in the slot/channel. Further, the apparatus comprises a laser system configured to fire a laser beam pulse of variable duration, via movable mirrors, which generates a linear projection of the otherwise stationery round spot. The length of such projected line is proportional to the width of the laser beam pulse. In an aspect, when the speeds of tablet in the rotating disk and the minor movement speeds are matched by hardware and software, the projected line of the laser beam produces a single spot/hole in the tablet. In an aspect, the depth of the hole is proportional to length of the laser pulse width. The length of the line generated by the minor movement as-well-as the depth can be controlled by the laser pulse power adjustment. The opening hole diameter of the hole is adjusted/settable by adjusting the focal lens ‘stand off’ distance above the tablet. In accordance with aspect of the present invention, the speed of the laser system drawing the lines is same as the speed of the rotating disk and the deflection of laser beam is preset. It is to be noted that the depth of the hole is proportional to the length of the line drawn. And the set width of line (by focusing/defocusing of the beam) decides its diameter. In accordance with the aspect of the present invention, the laser system may be configured to drill multiple holes by drawing a broken line, achieved by intermittent beam blanking within the pulse width of same pulse, in single rotation.

In another aspect, and feature, the tablets rotating on the disk guided by radial/spiraling slots/channels, are held in position without any mechanism to clamp them in fixed point for laser drilling operation, but are held locked at the end of the channel by virtue of the centrifugal force, thus making the unit far simpler to build and serviceable.

These and other features, aspects, and advantages of the present invention will become better understood with reference to the following description and appended claims.

### BRIEF DESCRIPTION OF DRAWINGS

The foregoing summary, as well as the following detailed description of preferred embodiments, is better understood when read in conjunction with the appended drawings. For the purpose of illustrating the invention, there is shown in the drawings example constructions of the invention; however, the invention is not limited to the specific systems disclosed in the drawings:

FIG. 1 shows isometric view of the high-speed laser drilling apparatus according to one exemplary embodiment of the invention.

FIG. 2 shows front view of the high-speed laser drilling apparatus according to one exemplary embodiment of the invention.

FIG. 3 shows exploded view of the rotary disk according to one exemplary embodiment of the invention.

FIG. 4 shows assembled view of the rotary disk according to one exemplary embodiment of the invention.

### DETAILED DESCRIPTION

Some embodiments of this invention, illustrating all its features, will now be discussed in detail.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present invention, the preferred, systems and methods are now described.

The disclosed embodiments are merely exemplary of the invention, which may be embodied in various forms.

The present invention relates to a laser drilling apparatus to precisely drill a hole in osmotic tablets with increased efficiency.

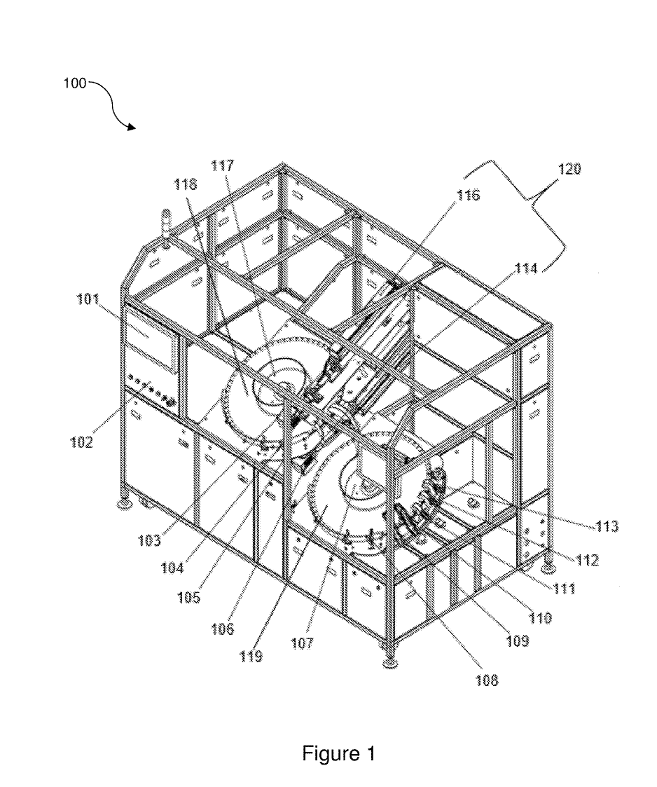

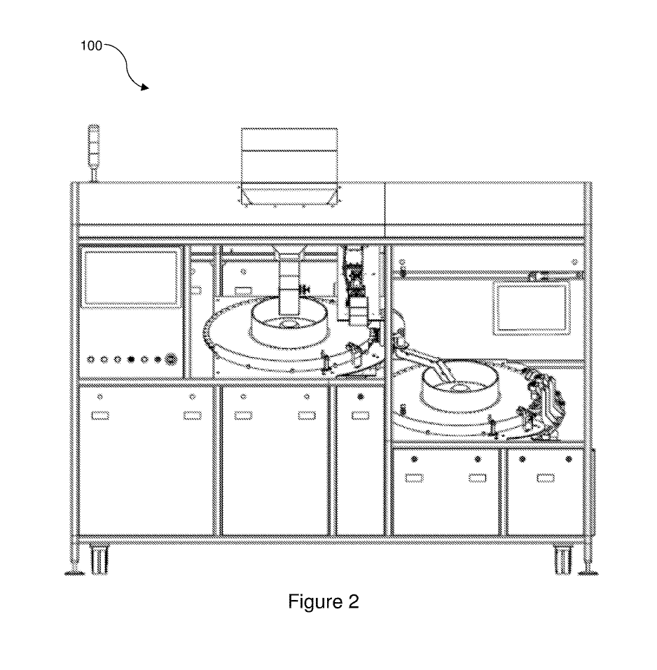

FIG. 1 shows an isometric view of the apparatus according to one exemplary embodiment of the invention. FIG. 2 illustrates the front view of the apparatus according to one exemplary embodiment of the invention. As illustrated in FIG. 1, the apparatus comprises a drilling section 117 which comprises of hopper and a rotary disk 118 comprising multiple radial slots. The tablets from hopper are sorted out on the rotary disk 118. Each radial slot holds one or more tablets. As the disk rotates at high speed, the rotary disk passes through colour sensors 104 and 105, and then through top galvo 103 and bottom galvo 106. Depending on the reading of the colour sensors 104 and 105, a top laser 116 or a bottom laser 114 is fired. The firing of the laser beam drills holes in the tablets contained in the radial slot. When the laser drilling is completed, it is ejected immediately by an air blow from bottom of the rotary disk and a chute on to another similar rotary disk 119 of the camera inspection section 107 as shown in FIG. 1. The camera inspection section 107 comprises a colour sensor 108 that identifies the surface of the tablet on which hole is made. Next, the cameras (109, 110) checks the quality of holes by comparing the circularity and diameter of the holes with the predefined reference values of the circularity and the diameter. Further, the cameras (109, 110) also checks location of the hole with respect to the geometrical centre of the tablet. Depending on the quality of holes produced, or presence of absence, of the hole the tablet enters the production chute 111 or the rejection chute 112. In case of bi layer tablets the colour sensor of this rotary disk in the inspection section diverts the tablet to recycle chute 113 and is collected in separate bin. HMI 101 and operation panel 102 are provided on the front side of the apparatus to control the apparatus. The HMI 101 is used for control data entry and to display errors, alarms and warnings to the user. The operation panel 102 (or HMI 102) displays the set up and camera set up for inspection parameters and on line images of running tablets with go no go messages in real time.

The tablets to be drilled are introduced into the hopper which is situated above the rotary disk. The controlled-release tablets are mostly manufactured with two different colours on two surfaces. The hole is drilled on one of the two surfaces. The drug is delivered by osmotic push technology. The layer below the drilled drug layer swells up when the tablet is introduced in liquid environment, it pushes the drug out from the drilled hole. The surface of tablets is coated with non-soluble bio-compatible coating and so drug is delivered only through the outlet that is provided by the drilled hole.

The tablets from hopper are transferred onto the rotary disk which has radial slots of required width. The width of these slots may be varied according to the size of the tablet. The rotary disks (118,119) can be easily replaced for different sized tablets. In accordance with an embodiment of the present invention, the time required for replacing the rotary disks is less than 5 minutes as compared to several hours taken by the conventional apparatuses available in the art. The tablets are lined up along the contour of the radial slots. The tablet on the circumference of the disk is the target tablet that must be laser drilled. The contour of the radial slots provides proper stability required to hold the tablet in place when it is being fired by the laser. The radial rotating disks (118,119) rotates at high speed and the resulting centrifugal force keeps the tablets flowing into the radial slots and holding them at their place. Such kind of design eliminates the need of any active or passive clamping required to hold the tablets.

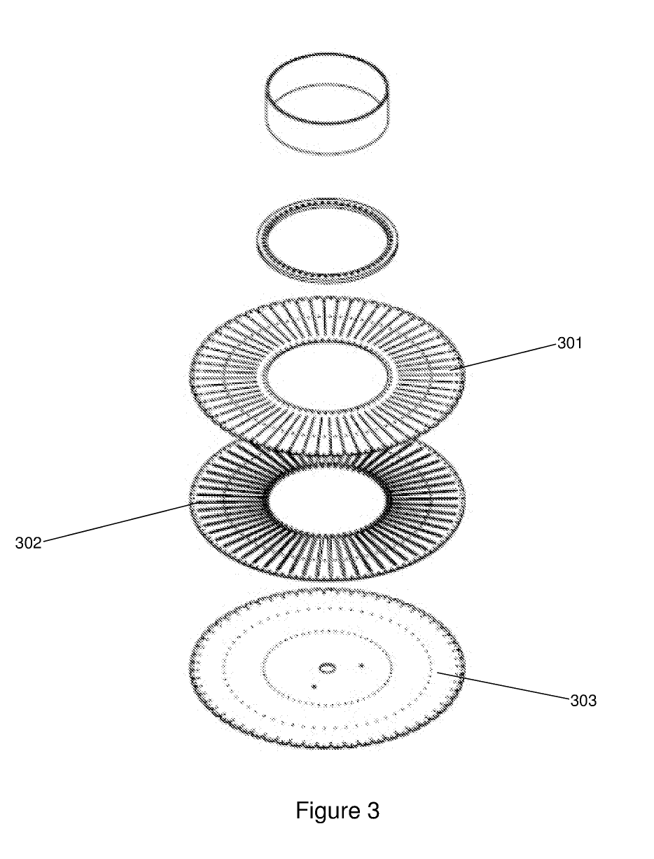

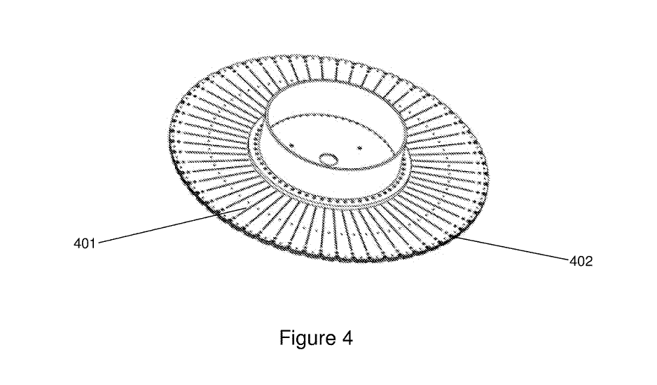

Each of the rotary disks (118,119) is an assembly of three plates 301, 302 and 303 as shown in FIG. 3. The plate 303 has semi-circular notches 402 (as shown in FIG. 4) on the circumference. When the target tablet reaches the circumference of the rotary disk 118, the notches enables the target tablet to be viewed from below the rotary disk 118 as well. Further, as shown in FIG. 4, each rotary disk has radial slots (401) to hold the tablets.

Now, again referring to FIG. 1, the rotary disk 118 is rotated at high speed and before passing through the laser system, the rotary disk 118 is passed through the colour sensors (104, 105). The colour sensor 104 is situated above the disk to determine the colour of the top surface of the tablet. The colour sensor 105 is situated beneath the rotary disk to sense the colour of the bottom surface of the tablet. Based on the colour sensed by either of the colour sensors (104, 105), it is decided whether the top laser 116 or the bottom laser 114 is to be fired. Further, as shown in FIG. 1, a galvo scanner 103 and a galvo scanner 106, placed at the top and the bottom of the rotary disk 118 respectively, is configured to assist the laser drilling operation. More particularly, the galvo scanner 103 and a galvo scanner 106 is configured to deflect the laser beam at a predefined trajectory of a line generated by software programming and synchronize the speed of the laser beam with the circumferential speed of the disk and hence the speed of the tablet.

According to embodiments of the present invention, the high-speed laser firing system fires the laser via the galvo mirrors (also referred as galvo scanners) in order to draw a line of length as per the pre-calibrated depth to length look up table. The drawing of line by the laser system has same speed as that of rotation of tablets on the disk. The diameter of the hole is decided by one time setting of the focus spot size by adjusting focal distance setting. This method ensures that the resulting hole maintains desired diameter. The circularity can be adjusted by adjusting the length of line and beam movement speed tuning, and laser on/off delay timings. In an embodiment, the diameter of the hole is within a range of 0.5 mm to 1 mm. For drilling such small holes, accurate circularity is difficult to maintain at high speed. Therefore, drilling by drawing the line proves to be efficient for making such holes as compared to drawing a circle profile on the tablet.

In accordance with embodiments of the present invention, the apparatus shown in FIG. 1 is further configured to drill multiple holes required to control time of the drug release by drawing spaced broken line. The apparatus enables drawing the of such broken lines just by laser beam blanking and accordingly drill multiple holes in a single rotation. When the laser is drawing a line, if there should be more than one holes, the laser is blanked, and thus two different holes at different places on the same tablet surface are formed.

Once all the above processes are completed in the drilling section, the tablets are passed on to the adjacent inspection section 107 where they are inspected individually by the cameras (109, 110). The inspection section comprises of identical rotary disk 119 as that of the drilling section. The camera 109 and the camera 110 may be positioned at the top and bottom of the identical rotary disk 119. Just before the tablets are passed through the cameras (109,110), the tablets are passed through a colour sensor 108. The colour sensor 108 is configured to sense the colour of the top surface of the tablets. Based on the colour sensed, one of the cameras (109,110) captures the snapshot of the tablet. The snap is analyzed and the placement of hole, the diameter, circularity and depth is checked. If the tablet passes the test, the tablet is taken up by the production chute 111. The tablets which are not perfectly drilled are either rejected or recycled through rejection chute 112 and recycle chute 113 respectively.

Thus, the apparatus of present invention works without any special complex arrangement to hold the tablets for drilling. Further, the apparatus facilitates in quick changing of rotary disk as per the requirements of the tablets to be drilled of varied sizes. Time required for changing the rotary disk is less than 5 minutes. The overall change part manufacturing cost of the apparatus disclosed therefore is minimal.

The preceding description has been presented with reference to various embodiments of the invention. However, Persons skilled in the art and technology to which this invention pertains will appreciate that alterations and changes in the described apparatus and methods of operation can be practiced without meaningfully departing from the principle, spirit and scope of this invention.

We claim:

1. An apparatus for drilling holes into a plurality of tablets, wherein the apparatus comprises:
a laser system (120) configured to fire a laser beam pulse, via movable mirrors, in order to generate a projected line, wherein the projected line enables in drilling a hole on desired side of a tablet based upon matching of the speed of the movable mirrors with the speed of a rotating disk (118) holding the tablet, and wherein the deflection of the laser beam pulse is preset.

2. The apparatus of claim 1, wherein each laser system (120) generates the projected line of thickness set based on focus spot size by setting focal distance setting.

3. The apparatus of claim 1, wherein the length of the projected line is set to match the diameter of the hole and control the circularity of the hole.

4. The apparatus of claim 1, wherein the laser system (120) is deployed either at the top of the rotary disk (118) or bottom of the rotary disk (118) or on both sides.

5. The apparatus of claim 1, wherein the laser system (120) is further configured to drill multiple holes by generating broken projected lines in a single rotation, wherein the multiple holes are drilled by blanking the laser beam.

6. The apparatus of claim 1, wherein the design and assembly contour of the radial slots of the rotary disk (118) enables accurate holding of one or more tablets.

7. The apparatus of claim 1, wherein the rotary disk (118) is replaced with a substitute rotary disk within a time period of 5 seconds in order to drill the tablets of varied sizes.

8. The apparatus of claim 1, wherein the length of the projected line is proportional to the width of the laser beam pulse.

9. The apparatus of claim 1, wherein the depth of the hole is proportional to the length of the laser pulse width.

10. A method for drilling holes into controlled release tablet at high speed using a laser system apparatus, the method comprising:
firing a laser beam pulse by the laser system (120), via movable mirrors, in order to generate a projected line, wherein the projected line enables in drilling a hole on desired side of a tablet based upon matching of the speed of the movable mirrors with the speed of a rotating disk (118) holding the tablet, and wherein the deflection of the laser beam is preset.

11. The method of claim 10, wherein the multiple holes are drilled by generating broken projected lines in a single rotation, wherein the multiple holes are drilled by blanking the laser beam.

12. The method of claim 10, wherein the laser system (120) is fired after locating the desired side of tablet surface to be drilled.

13. The method of claim 10, wherein the length of the projected line is proportional to the width of the laser beam pulse.

14. The method of claim 10, wherein the depth of the hole is proportional to the length of the laser pulse width.
